(19) United States
(12) Patent Application Publication
(10) Pub. No.: US 2024/0410869 A1
(43) Pub. Date: Dec. 12, 2024

(54) SENSOR

(71) Applicant: KABUSHIKI KAISHA TOSHIBA, Tokyo (JP)

(72) Inventors: Ping WANG, Fujisawa Kanagawa (JP); Masayuki MATSUTAKE, Yokohama Kanagawa (JP); Hiroaki YAMAZAKI, Yokohama Kanagawa (JP); Yoshihiko KURUI, Chigasaki Kanagawa (JP); Yosuke AKIMOTO, Yokohama Kanagawa (JP)

(73) Assignee: KABUSHIKI KAISHA TOSHIBA, Tokyo (JP)

(21) Appl. No.: 18/583,115

(22) Filed: Feb. 21, 2024

(30) Foreign Application Priority Data
Jun. 7, 2023 (JP) 2023-094132

(51) Int. Cl. G01N 33/00; G01N 25/18

(52) U.S. Cl. CPC G01N 33/0073; G01N 25/18

(57) ABSTRACT

According to one embodiment, a sensor includes a sensor section and a circuit section. The sensor section includes a first element portion including a first resistance element and a first conductive member, and a second element portion including a second resistance element. The circuit section includes a detector and a controller, and is configured to perform a first operation. The controller controls a first power in the first operation based on a second signal corresponding to a second resistance of the second resistance element. In the first operation: the controller supplies the first power to the first conductive member to increase a first temperature of the first element section; and the detector outputs a detected value corresponding to a difference between the second signal and a first signal corresponding to a first resistance of the first resistance element in a first state in which the first temperature is raised.

### CROSS-REFERENCE TO RELATED APPLICATIONS

This application is based upon and claims the benefit of priority from Japanese Patent Application No. 2023-094132, filed on Jun. 7, 2023; the entire contents of which are incorporated herein by reference.

### FIELD

Embodiments described herein generally relate to a sensor.

### BACKGROUND

For example, there are sensors using MEMS (Micro Electro Mechanical Systems) elements. It is desired to improve the characteristics of the sensor.

### BRIEF DESCRIPTION OF THE DRAWINGS

FIG. 1 is a block diagram illustrating a sensor according to a first embodiment;

FIG. 2 is a schematic cross-sectional view illustrating the sensor according to the first embodiment;

FIG. 3 is a schematic plan view illustrating the sensor according to the first embodiment;

FIG. 4 is a schematic plan view illustrating the sensor according to the first embodiment;

FIGS. 5A to 5E are schematic diagrams illustrating the operation of the sensor according to the first embodiment;

FIG. 6 is a schematic cross-sectional view illustrating a sensor according to the first embodiment;

FIG. 7 is a block diagram illustrating a sensor according to a second embodiment; and

FIG. 8 is a schematic cross-sectional view illustrating the sensor according to the second embodiment.

### DETAILED DESCRIPTION

According to one embodiment, a sensor includes a sensor section and a circuit section. The sensor section includes a first element portion including a first resistance element and a first conductive member, and a second element portion including a second resistance element. The circuit section includes a detector and a controller. The circuit section is configured to perform a first operation. The controller is configured to control a first power in the first operation based on a second signal corresponding to a second resistance of the second resistance element. In the first operation, the controller is configured to supply the first power to the first conductive member to increase a first temperature of the first element section. In the first operation, the detector is configured to output a detected value corresponding to a difference between the second signal and a first signal corresponding to a first resistance of the first resistance element in a first state in which the first temperature is raised.

Various embodiments are described below with reference to the accompanying drawings.

The drawings are schematic and conceptual; and the relationships between the thickness and width of portions, the proportions of sizes among portions, etc., are not necessarily the same as the actual values. The dimensions and proportions may be illustrated differently among drawings, even for identical portions.

In the specification and drawings, components similar to those described previously in an antecedent drawing are marked with like reference numerals, and a detailed description is omitted as appropriate.

### First Embodiment

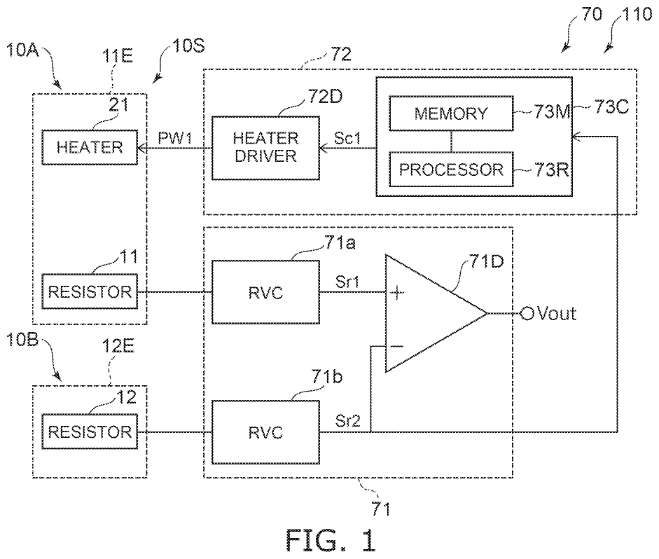

FIG. 1 is a block diagram illustrating a sensor according to a first embodiment.

As shown in FIG. 1, a sensor 110 according to the embodiment includes a sensor section 10S and a circuit section 70.

The sensor section 10S includes a first element portion 11E and a second element portion 12E. The first element portion 11E includes a first resistance element 11 and a first conductive member 21. The second element portion 12E includes a second resistance element 12. As described later, the second element portion 12E may include a second conductive member 22. The first element portion 11E is included in a first detection element 10A. The second element portion 12E is included in a second detection element 10B.

The circuit section 70 includes a detector 71 and a controller 72. The circuit section 70 is configured to perform a first operation. In the first operation, the controller 72 can control a first power PW1 based on a second signal Sr2 corresponding to a second resistance of the second resistance element 12. In the first operation, the controller 72 can supply the first power PW1 being controlled to the first conductive member 21 to increase a first temperature of the first element portion 11E. In the first operation, the detector 71 can output the detected value Vout corresponding to a difference between the second signal Sr2 and the first signal Sr1 corresponding to the first resistance of the first resistance element 11 in the first state in which the first temperature is raised. The first power PW1 may be a voltage value.

The first conductive member 21 is heated by the first power PW1. The first conductive member 21 is, for example, a heater. The heat of the first conductive member 21 increases the temperature of the first element portion 11E. As a result, the temperature of the first resistance element 11 increases.

The state of the temperature of the first resistance element 11 depends on the state of a detection target around the sensor section 10S (for example, the first element portion 11E). The detection target is, for example, gas. The gas is, for example, hydrogen or carbon dioxide. Thermal conductivity (heat dissipation) changes depending on the type of gas and the concentration of the gas. Due to the change in heat dissipation, the temperature state of the first resistance element 11 corresponds to the state of the detection target. By detecting the temperature of the first resistance element 11, the state of the detection target (such as the type and concentration of gas) can be detected. The sensor 110 is, for example, a thermally conductive gas sensor.

In the embodiment, the difference between the second signal Sr2 and the first signal Sr1 corresponding to the first resistance of the first resistance element 11 is detected. Thereby, the detection target can be detected with higher accuracy. The second resistance element 12 is, for example, a reference element.

Thus, the first signal Sr1 changes depending on the detection target around the sensor section 10S. On the other hand, the second signal Sr2 does not change depending on the detection target. The change rate of the second signal Sr2 with respect to the detection target is lower than the change rate of the first signal Sr1 with respect to the detection target.

In this example, a signal obtained from the first resistance element 11 is converted into a voltage by the first resistance voltage conversion circuit 71a (RVC). The voltage obtained by the conversion corresponds to the first signal Sr1. A signal obtained from the second resistance element 12 is converted into a voltage by the second resistance voltage conversion circuit 71b. A voltage obtained by the conversion corresponds to the second signal Sr2. These signals are input to the differential circuit 71D. The detected value Vout corresponding to the difference is output from the differential circuit 71D.

In the detection operation as described above, a reference example in which the first power PW1 is constant can be considered. In this reference example, for example, when the environmental temperature of the sensor section 10S changes, if the first power PW1 being constant is supplied to the first conductive member 21 (heater), a state in which the temperature of the first conductive member 21 may not be the desired temperature may occur. Thereby, the accuracy of detection results may not be sufficiently improved.

In the embodiment, the first power PW1 is controlled based on the temperature detection result of the sensor section 10S. As shown in FIG. 1, in the case of the sensor 110, the second signal Sr2 corresponding to the second resistance of the second resistance element 12 is supplied to the controller 72. The controller 72 controls the first power PW1 based on the second signal Sr2. The second resistance of the second resistance element 12 corresponds to the environmental temperature of the sensor section 10S. By controlling the first power PW1 using the second signal Sr2 corresponding to the second resistance of the second resistance element 12, the temperature of the first conductive member 21 can be brought to the desired state with high accuracy. Thereby, the detection target can be detected with high accuracy. According to the embodiment, it is possible to provide a sensor whose characteristics can be improved.

As shown in FIG. 1, in this example, the controller 72 includes a drive circuit 72D and a processing circuit 73C. The drive circuit 72D is configured to supply the first power PW1 to the first conductive member 21. The drive circuit 72D may be configured to apply a voltage corresponding to the first power PW1 to the first conductive member 21. The processing circuit 73C is configured to control the first power PW1 (or voltage) by controlling the drive circuit 72D based on the second signal Sr2.

For example, the drive circuit 72D may include circuitry for generating a standard voltage. The processing circuit 73C may generate a correction voltage according to the second signal Sr2. The first power PW1 (voltage) may be controlled by adding the standard voltage and the correction voltage.

As shown in FIG. 1, in one example, the processing circuit 73C includes a processor 73R and a memory 73M. The memory 73M is configured to store the relationship between the change in the second signal Sr2 and the control value regarding the first power PW1. For example, the memory 73M may be configured to store a table regarding these relationships. For example, the memory 73M may be configured to store a function related to these relationships. The processor 73R is configured to control the drive circuit 72D based on the above relationship stored in the memory 73M and the second signal Sr2 supplied to the controller 72 (for example, the processing circuit 73C). For example, a control signal Sc1 is supplied from the processor 73R to the drive circuit 72D.

The second signal Sr2 obtained from the second resistance element 12 changes depending on the second temperature around the sensor section 10S. The second resistance element 12 functions as a temperature sensor. The first power PW1 changes according to the second temperature (for example, environmental temperature).

An example of the sensor section 10S will be described below.

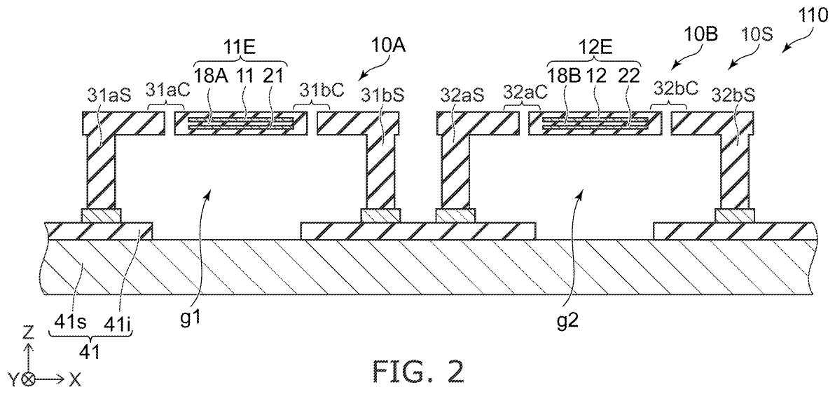

FIG. 2 is a schematic cross-sectional view illustrating the sensor according to the first embodiment.

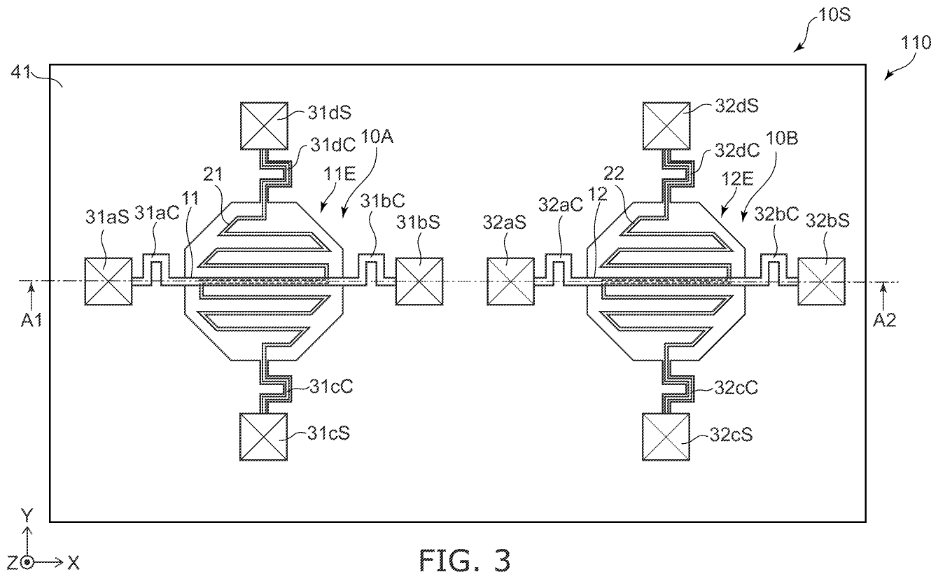

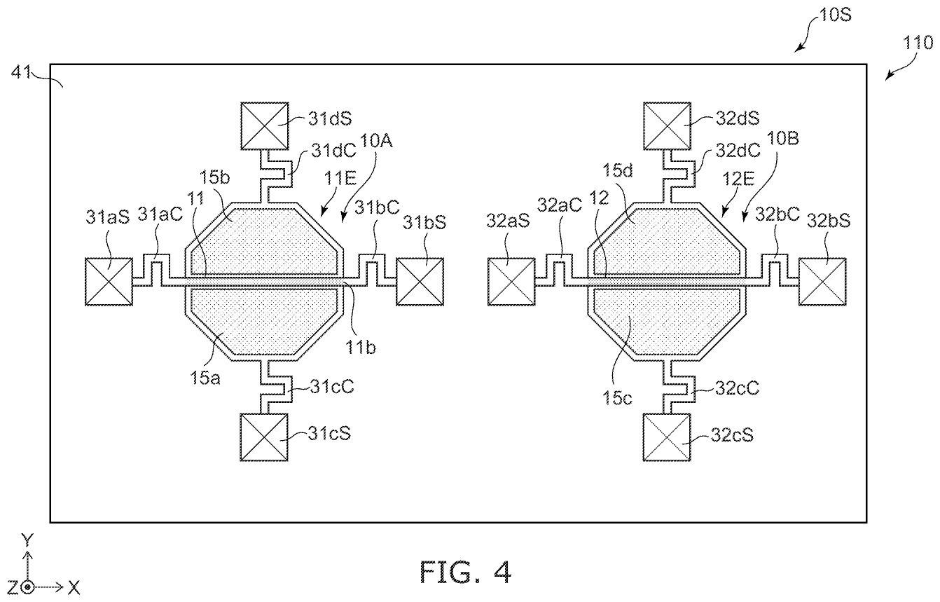

FIGS. 3 and 4 are schematic plan views illustrating the sensor according to the first embodiment.

FIG. 2 is a sectional view taken along the line A1-A2 in FIG. 3.

As shown in FIG. 2, the sensor section 10S further includes a base 41. The first element portion 11E and the second element portion 12E are fixed to the base 41. By providing these element portions in base 41, the temperatures of these element portions become substantially the same when power is not supplied to the first conductive member 21. Thereby, higher precision detection is possible in detection using the second resistance element 12 as a reference.

The control of the first power PW1 is performed based on the second signal Sr2 corresponding to the second resistance of the second resistance element 12. The second element portion 12E including the second resistance element 12 is spatially close to the first element portion 11E. The first power PW1 is controlled with good spatial responsiveness. In the embodiment, the heat capacity of the first element portion 11E is small. The temperature of the first element portion 11E can be changed in a short time. The first power PW1 can be controlled with high temporal responsiveness.

As shown in FIG. 2, the base 41 may include a semiconductor substrate 41s and an insulating layer 41i. The semiconductor substrate 41s may be, for example, a silicon substrate. The insulating layer 41i is provided on the semiconductor substrate 41s. A part of the semiconductor substrate 41s may include a transistor or the like. At least a part of the circuit section 70 may be formed by a part of the semiconductor substrate 41s.

As shown in FIG. 2, the sensor section 10S includes a first support portion 31aS and a second support portion 32aS. The first support portion 31aS and the second support portion 32aS are fixed to the base 41. The first support portion 31aS supports the first element portion 11E. A first gap g1 is provided between the base 41 and the first element portion 11E. The second support portion 32aS supports the second element portion 12E. A second gap g2 is provided between the base 41 and the second element portion 12E. By providing these gaps, for example, influence from the outside via the base 41 is suppressed. It is easier to obtain higher accuracy.

The first support portion 31aS is included in the first detection element 10A. The second support portion 32aS is included in the second detection element 10B. In this example, the first detection element 10A includes a first connection portion 31aC. The first connecting portion 31aC connects the first element portion 11E to the first support portion 31aS. The second detection element 10B includes a second connection portion 32aC. The second connecting portion 32aC connects the second element portion 12E to the second support portion 32aS. These connecting portions may have a meandering structure, for example. These connection portions provide low thermal conductivity. For example, the first element portion 11E can be efficiently heated.

The first element portion 11E includes a first insulating member 18A. At least a part of the first insulating member 18A is provided between the first conductive member 21 and the first resistance element 11.

In this example, the second element portion 12E includes a second conductive member 22. For example, the configuration of the second element portion 12E is the same as the configuration of the first element portion 11E. For example, the thermal characteristics (heat capacity, etc.) of the second element portion 12E are substantially the same as the thermal characteristics (heat capacity, etc.) of the first element portion 11E. Correction with higher accuracy is possible. The second conductive member 22 does not need to be supplied with power. The second element portion 12E includes a second insulating member 18B. At least a part of the second insulating member 18B is provided between the second conductive member 22 and the second resistance element 12.

In this example, the sensor section 10S includes a first opposing support portion 31bS and a second opposing support portion 32bS. The first opposing support portion 31bS and the second opposing support portion 32bS are fixed to the base 41. The first opposing support portion 31bS supports the first element portion 11E. The second opposing support portion 32bS supports the second element portion 12E. The element portions are held more stably.

In this example, the first detection element 10A includes a first opposing connection portion 31bC. The first opposing connection portion 31bC connects the first element portion 11E to the first opposing support portion 31bS. The second detection element 10B includes a second opposing connection portion 32bC. The second opposing connection portion 32bC connects the second element portion 12E to the second opposing support portion 32bS.

As shown in FIG. 3, the first detection element 10A may include a first other support portion 31cS, a first other connection portion 31cC, a first other opposing support portion 31dS, and a first other opposing connection portion 31dC. The first other support portion 31cS and the first other opposing support portion 31dS are fixed to the base 41. The first other connecting portion 31cC connects the first element portion 11E to the first other support portion 31cS. The first other opposing connection portion 31dC connects the first element portion 11E to the first other opposing support portion 31dS.

As shown in FIG. 3, the second detection element 10B may include a second other support portion 32cS, a second other connection portion 32cC, a second other opposing support portion 32dS, and a second other opposing connection portion 32dC. The second other support portion 32cS and the second other opposing support portion 32dS are fixed to the base 41. The second other connecting portion 32cC connects the second element portion 12E to the second other support portion 32cS. The second other opposing connection portion 32dC connects the second element portion 12E to the second other opposing support portion 32dS.

In this example, the first resistance element 11 is electrically connected to the outside (for example, the circuit section 70) via the first connection portion 31aC and the first opposing connection portion 31bC. The first conductive member 21 is electrically connected to the outside (for example, the circuit section 70) via the first other connection portion 31cC and the first other opposing connection portion 31dC.

In this example, the second resistance element 12 is electrically connected to the outside (for example, the circuit section 70) via the second connection portion 32aC and the second opposing connection portion 32bC. The second conductive member 22 does not need to be electrically connected to the outside (for example, the circuit section 70).

FIG. 4 illustrates a pattern shape of a layer including the first resistance element 11 and the second resistance element 12. As shown in FIG. 4, a first film 15a and a second film 15b may be provided. The first resistance element 11 is provided between the first film 15a and the second film 15b. The materials of these films may be the same as the material of the first resistance element 11. A third film 15c and a fourth film 15d may be provided. The second resistance element 12 is provided between the third film 15c and the fourth film 15d. The materials of these films may be the same as the material of the second resistance element 12. These films suppress deformation of the first element portion 11E and the second element portion 12E, for example.

FIGS. 5A to 5E are schematic diagrams illustrating the operation of the sensor according to the first embodiment.

These figures are time charts illustrating the first operation. The horizontal axis of these figures is time tm. The vertical axis in FIG. 5A is a value PW1 (t) of the first power PW1. The vertical axis in FIG. 5B is a value Sr1 (t) of the first signal Sr1. The vertical axis in FIG. 5C is a value Sr2 (t) of the second signal Sr2. The vertical axis in FIG. 5D is a value Vout (t) of the detected value Vout. The vertical axis in FIG. 5E is a value Sc1 (t) of the control signal Sc1.

As shown in FIG. 5A, for example, pulses of the first power PW1 are repeatedly output. The pulses may be output repeatedly in the first period T1. One pulse corresponds to one first operation. As shown in FIG. 5B, the first signal Sr1 varies depending on the pulse of the first power PW1. On the other hand, as shown in FIG. 5C, for example, the second signal Sr2 changes in response to a change in the ambient temperature. Based on the first signal Sr1 and the second signal Sr2, the detected value Vout corresponding to the difference between these signals is calculated. The detected value Vout corresponds to, for example, the detection result of the detection target. The detected value Vout may be an analog value or a digital value. The detected value Vout is output during one first period T1.

As shown in FIG. 5E, the control signal Sc1 is output. The control signal Sc1 is based on the second signal Sr2, for example. The control signal Sc1 may be an analog signal or a digital signal. The next pulse of the first power PW1 is controlled based on control signal Sc1. Such operations may be performed repeatedly.

In this way, the circuit section 70 may be configured to repeatedly perform the first operation. The repetition has the first period T1. For example, the first period T1 includes a first time duration Tp1 and a second time duration Tp2 after the first time duration Tp1. In this example, in the first time duration Tp1, the controller 72 supplies the first power PW1 to the first conductive member 21, and the detector 71 outputs the detected value Vout. The controller 72 controls the first power PW1 based on the second signal Sr2 in the second period Tp2. For example, the first time duration Tp1 may be longer than the second time duration Tp2. By the first time duration Tp1 being long, for example, the time for detecting the detection target can be sufficiently long, and it becomes easy to obtain highly accurate detection results. By the second time duration Tp2 being short, the first period T1 can be shortened. High frequency detection operations can be performed.

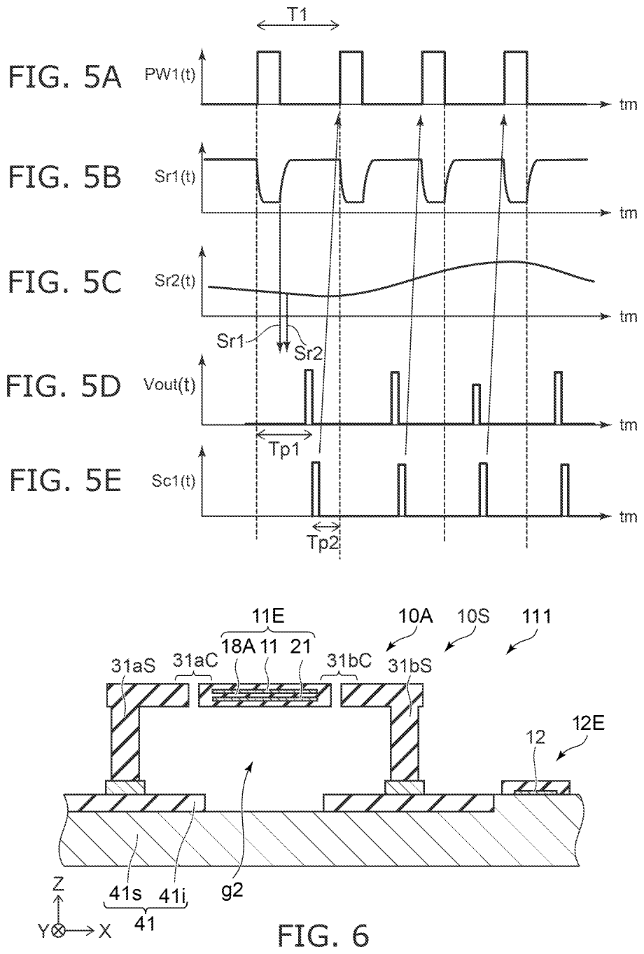

FIG. 6 is a schematic cross-sectional view illustrating a sensor according to the first embodiment.

As shown in FIG. 6, a sensor 111 according to the embodiment also includes the sensor section 10S and the circuit section 70 (see FIG. 1). The sensor section 10S includes the first element portion 11E and the second element portion 12E. The first element portion 11E includes the first resistance element 11 and the first conductive member 21. The second element portion 12E includes the second resistance element 12. The circuit section 70 includes the detector 71 and the controller 72 (see FIG. 1).

In the sensor 111, the first element portion 11E has a MEMS structure. The second element portion 12E is fixed to the base 41, and no gap is provided between the base 41 and the second element portion 12E.

Also in the sensor 111, the circuit section 70 is configured to perform the first operation (see FIG. 1) described with respect to the sensor 110. For example, the controller 72 is configured to control the first power PW1 based on the second signal Sr2 corresponding to the second resistance of the second resistance element 12 in the first operation. In the first operation, the controller 72 supplies the first power PW1 being controlled to the first conductive member 21 to raise the first temperature of the first element portion 11E. In the first operation, the detector 71 is configured to output the detected value Vout corresponding to the difference between the second signal Sr2 and the first signal Sr1 corresponding to the first resistance of the first resistance element 11 in the first state in which the first temperature is raised. The sensor 111 can also detect the detection target with high accuracy. According to the embodiment, a sensor capable of improving characteristics can be provided.

### Second Embodiment

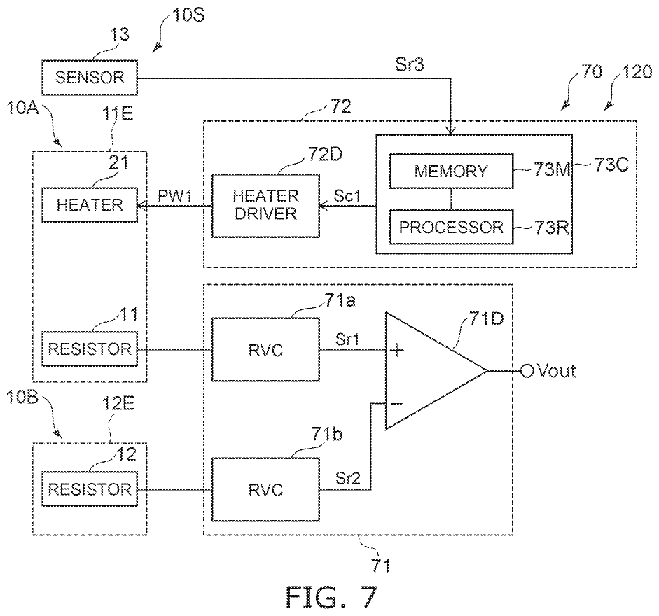

FIG. 7 is a block diagram illustrating a sensor according to a second embodiment.

As shown in FIG. 7, a sensor 120 according to the embodiment includes the sensor section 10S and the circuit section 70. In the sensor 120, the sensor section 10S includes a temperature sensor 13. The configuration of the sensor 120 other than this may be the same as the configuration of the sensor 110.

In the sensor 120, the sensor section 10S includes the first element portion 11E, the second element portion 12E, and the temperature sensor 13. The first element portion 11E includes the first resistance element 11 and the first conductive member 21. The second element portion 12E includes the second resistance element 12. The second element portion 12E may include the second conductive member 22.

In the sensor 120, the circuit section 70 includes the detector 71 and the controller 72. The circuit section 70 is configured to perform the first operation. In the first operation, the controller 72 is configured to control the first power PW1 based on a detection signal Sr3 of the temperature sensor 13. In the first operation, the controller 72 is configured to supply the first power PW1 being controlled to the first conductive member 21 to increase the first temperature of the first element portion 11E. In the first operation, the detector 71 is configured to output the detected value Vout corresponding to the difference between the first signal Sr1 corresponding to the first resistance of the first resistance element 11 in the first state in which the first temperature is raised and the second signal Sr2 corresponding to the second resistance of the second resistance element 12.

In the sensor 120, the first power PW1 is controlled using the detection signal Sr3 of the temperature sensor 13. Thereby, the temperature of the first conductive member 21 can be brought to a desired state with high accuracy. Thereby, the detection target can be detected with high accuracy. According to the embodiment, it is possible to provide a sensor whose characteristics can be improved.

The controller 72 may include the drive circuit 72D and the processing circuit 73C. The drive circuit 72D is configured to supply the first power PW1 to the first conductive member 21. The processing circuit 73C is configured to control the first power PW1 by controlling the drive circuit 72D based on the detection signal Sr3. For example, the control signal Sc1 is supplied from the processor 73R to the drive circuit 72D.

For example, the processing circuit 73C may include the processor 73R and the memory 73M. The memory 73M is configured to store the relationship between the change in the detection signal Sr3 and the control value regarding the first power PW1. The processor 73R is configured to control the drive circuit 72D based on the above relationship stored in the memory 73M and the detection signal Sr3 supplied to the controller 72 (for example, the processing circuit 73C).

In the sensor 120, the detection signal Sr3 changes depending on the second temperature around the sensor section 10S. For example, the first power PW1 changes depending on the second temperature.

For example, the first signal Sr1 corresponding to the first resistance element 11 changes depending on the detection target (for example, the state of the detection target gas) around the sensor section 10S. The second signal Sr2 does not change depending on the detection target. Alternatively, the change rate of the second signal Sr2 with respect to the detection target is lower than the change rate of the first signal Sr1 with respect to the detection target.

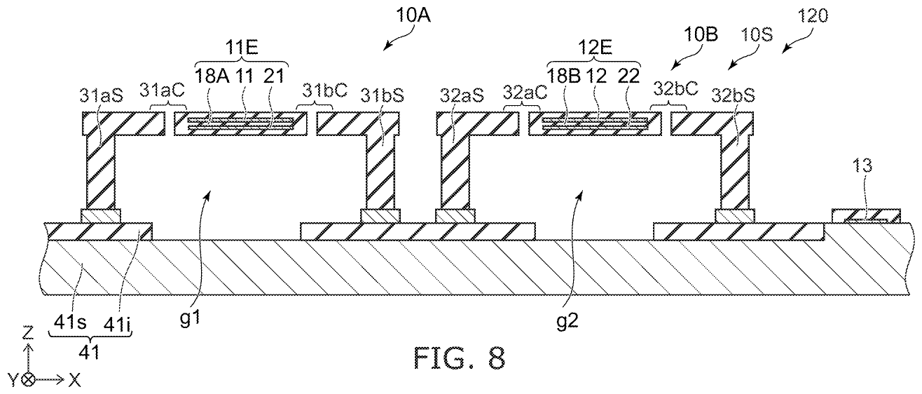

FIG. 8 is a schematic cross-sectional view illustrating the sensor according to the second embodiment.

As shown in FIG. 8, in the sensor 120, the sensor section 10S further includes the base 41. The first element portion 11E, the second element portion 12E, and the temperature sensor 13 are fixed to the base 41. Control with high spatial responsiveness becomes possible. Any element can be used as the temperature sensor 13.

For example, the sensor section 10S includes the first support portion 31aS fixed to the base 41 and the second support portion 32aS fixed to the base 41. The first support portion 31aS supports the first element portion 11E. The first gap g1 is provided between the base 41 and the first element portion 11E. The second support portion 32aS supports the second element portion 12E. The second gap g2 is provided between the base 41 and the second element portion 12E. The first conductive member 21 can be heated efficiently. Thermal influence from the outside via the base 41 and the like can be suppressed. Detection with higher accuracy becomes possible.

The embodiments may include the following configurations (technical proposals).

### (Configuration 1)

A sensor, comprising:
a sensor section including a first element portion, and a second element portion, the first element portion including a first resistance element and a first conductive member, the second element portion including a second resistance element; and
a circuit section including a detector and a controller,
the circuit section being configured to perform a first operation,
the controller being configured to control a first power in the first operation based on a second signal corresponding to a second resistance of the second resistance element,
in the first operation, the controller being configured to supply the first power to the first conductive member to increase a first temperature of the first element section, and
in the first operation, the detector being configured to output a detected value corresponding to a difference between the second signal and a first signal corresponding to a first resistance of the first resistance element in a first state in which the first temperature is raised.

### (Configuration 2)

The sensor according to Configuration 1, further comprising:
a base,
the first element portion and the second resistance element being fixed to the base.

### (Configuration 3)

The sensor according to Configuration 2, wherein
the sensor section further includes
   a first support portion fixed to the base, and
   a second support portion fixed to the base, the first support portion supports the first element portion,
a first gap is provided between the base and the first element portion,
the second support portion supports the second element portion, and
a second gap is provided between the base and the second element portion.

### (Configuration 4)

The sensor according to any one of Configurations 1-3, wherein
the controller includes a drive circuit and a processing circuit,
the drive circuit is configured to supply the first power to the first conductive member, and
the processing circuit is configured to control the first power by controlling the drive circuit based on the second signal.

### (Configuration 5)

The sensor according to Configuration 4, wherein
the processing circuit includes a processor and a memory,
the memory is configured to store a relationship between a change in the second signal and a control value regarding the first power, and
the processor is configured to control the drive circuit based on the relationship stored in the memory and the second signal supplied to the controller.

### (Configuration 6)

The sensor according to any one of Configurations 1-5, wherein
the second signal changes depending on a second temperature around the sensor section, and
the first power changes depending on the second temperature.

### (Configuration 7)

The sensor according to any one of Configurations 1-6, wherein
the first signal changes depending on a detection target around the sensor section, and
the second signal does not change depending on the detection target, or a change rate of the second signal with respect to the detection target is lower than a change rate of the first signal with respect to the detection target.

### (Configuration 8)

The sensor according to any one of Configurations 1-7, wherein
the circuit section is configured to perform repeating the first operation,
the repeating has a first period,
the first period includes a first time duration and a second time duration after the first time duration,
in the first time period, the controller supplies the first electric power to the first conductive member, and the detector outputs the detected value,
in the second time duration, the controller controls the first power based on the second signal, and
the first time duration is longer than the second time duration.

### (Configuration 9)

A sensor, comprising:
a sensor section including a first element portion, a second element portion, and a temperature sensor, the first element portion including a first resistance element and a first conductive member, the second element portion including a second resistance element; and
a circuit section including a detector and a controller,
the circuit section being configured to perform a first operation,
in the first operation, the controller being configured to control a first power based on a detection signal of the temperature sensor,
in the first operation, the controller being configured to supply the first power to the first conductive member to increase a first temperature of the first element portion, and
in the first operation, the detector being configured to output a detected value corresponding to a difference between a first signal corresponding to a first resistance of the first resistance element in a first state in which the first temperature is raised and the second signal corresponding to a second resistance of the second resistance element.

### (Configuration 10)

The sensor according to Configuration 9, further comprising:
a base,
the first element portion, the second element portion and the temperature sensor being fixed to the base.

### (Configuration 11)

The sensor according to Configuration 10, wherein
the sensor section further includes
   a first support portion fixed to the base, and
   a second support portion fixed to the base,
the first support portion supports the first element portion,
a first gap is provided between the base and the first element portion,
the second support portion supports the second element portion, and
a second gap is provided between the base and the second element portion.

### (Configuration 12)

The sensor according to any one of Configurations 9-11, wherein
the controller includes a drive circuit and a processing circuit,
the drive circuit is configured to supply the first power to the first conductive member, and
the processing circuit is configured to control the first power by controlling the drive circuit based on the detection signal.

### (Configuration 13)

The sensor according to Configuration 12, wherein
the processing circuit includes a processor and a memory,
the memory is configured to store a relationship between a change in the detection signal and a control value regarding the first power, and
the processor is configured to control the drive circuit based on the relation stored in the memory and the detection signal supplied to the controller.

### (Configuration 14)

The sensor according to any one of Configurations 9-13, wherein
the detection signal changes depending on a second temperature around the sensor section, and
the first power changes depending on the second temperature.

### (Configuration 15)

The sensor according to any one of Configurations 9-14, wherein
the first signal changes depending on a detection target around the sensor section, and
the second signal does not change depending on the detection target, or a change rate of the second signal with respect to the detection target is lower than a change rate of the first signal with respect to the detection object.

According to the embodiment, a sensor with improved characteristics can be provided.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in the sensors such as bases, sensor sections, control sections, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all sensors practicable by an appropriate design modification by one skilled in the art based on the sensors described above as embodiments of the invention also are within the scope of the invention to the extent that the purport of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor, comprising:
a sensor section including a first element portion, and a second element portion, the first element portion including a first resistance element and a first conductive member, the second element portion including a second resistance element; and
a circuit section including a detector and a controller,
the circuit section being configured to perform a first operation,
the controller being configured to control a first power in the first operation based on a second signal corresponding to a second resistance of the second resistance element,
in the first operation, the controller being configured to supply the first power to the first conductive member to increase a first temperature of the first element section, and
in the first operation, the detector being configured to output a detected value corresponding to a difference between the second signal and a first signal corresponding to a first resistance of the first resistance element in a first state in which the first temperature is raised.

2. The sensor according to claim 1, further comprising:
a base,
the first element portion and the second resistance element being fixed to the base.

3. The sensor according to claim 2, wherein
the sensor section further includes
a first support portion fixed to the base, and
a second support portion fixed to the base,
the first support portion supports the first element portion,
a first gap is provided between the base and the first element portion,
the second support portion supports the second element portion, and
a second gap is provided between the base and the second element portion.

4. The sensor according to claim 1, wherein
the controller includes a drive circuit and a processing circuit,
the drive circuit is configured to supply the first power to the first conductive member, and
the processing circuit is configured to control the first power by controlling the drive circuit based on the second signal.

5. The sensor according to claim 4, wherein
the processing circuit includes a processor and a memory,
the memory is configured to store a relationship between a change in the second signal and a control value regarding the first power, and
the processor is configured to control the drive circuit based on the relationship stored in the memory and the second signal supplied to the controller.

6. The sensor according to claim 1, wherein
the second signal changes depending on a second temperature around the sensor section, and
the first power changes depending on the second temperature.

7. The sensor according to claim 1, wherein
the first signal changes depending on a detection target around the sensor section, and
the second signal does not change depending on the detection target, or a change rate of the second signal with respect to the detection target is lower than a change rate of the first signal with respect to the detection target.

8. The sensor according to claim 1, wherein
the circuit section is configured to perform repeating the first operation,
the repeating has a first period,
the first period includes a first time duration and a second time duration after the first time duration,
in the first time period, the controller supplies the first electric power to the first conductive member, and the detector outputs the detected value,
in the second time duration, the controller controls the first power based on the second signal, and
the first time duration is longer than the second time duration.

9. A sensor, comprising:
a sensor section including a first element portion, a second element portion, and a temperature sensor, the first element portion including a first resistance element and a first conductive member, the second element portion including a second resistance element; and
a circuit section including a detector and a controller,
the circuit section being configured to perform a first operation,
in the first operation, the controller being configured to control a first power based on a detection signal of the temperature sensor,
in the first operation, the controller being configured to supply the first power to the first conductive member to increase a first temperature of the first element portion, and
in the first operation, the detector being configured to output a detected value corresponding to a difference between a first signal corresponding to a first resistance of the first resistance element in a first state in which the first temperature is raised and the second signal corresponding to a second resistance of the second resistance element.

10. The sensor according to claim 9, further comprising:
a base,
the first element portion, the second element portion and the temperature sensor being fixed to the base.

11. The sensor according to claim 10, wherein
the sensor section further includes
a first support portion fixed to the base, and
a second support portion fixed to the base,
the first support portion supports the first element portion,
a first gap is provided between the base and the first element portion,
the second support portion supports the second element portion, and
a second gap is provided between the base and the second element portion.

12. The sensor according to claim 9, wherein
the controller includes a drive circuit and a processing circuit,
the drive circuit is configured to supply the first power to the first conductive member, and
the processing circuit is configured to control the first power by controlling the drive circuit based on the detection signal.

13. The sensor according to claim 12, wherein
the processing circuit includes a processor and a memory,
the memory is configured to store a relationship between a change in the detection signal and a control value regarding the first power, and
the processor is configured to control the drive circuit based on the relation stored in the memory and the detection signal supplied to the controller.

14. The sensor according to claim 9, wherein
the detection signal changes depending on a second temperature around the sensor section, and
the first power changes depending on the second temperature.

15. The sensor according to claim 9, wherein
the first signal changes depending on a detection target around the sensor section, and
the second signal does not change depending on the detection target, or a change rate of the second signal with respect to the detection target is lower than a change rate of the first signal with respect to the detection object.
